(19) United States
(12) Patent Application Publication
(10) Pub. No.: US 2016/0312233 A1
(43) Pub. Date: Oct. 27, 2016

(54) NEW METHOD OF SELECTION OF ALGAL-TRANSFORMED CELLS USING NUCLEASE

(71) Applicant: CELLECTIS, Paris (FR)

(72) Inventors: Philippe DUCHATEAU, Draveil (FR); Fayza DABOUSSI, Chelles (FR)

(21) Appl. No.: 15/103,751

(22) PCT Filed: Dec. 12, 2014
(86) PCT No.: PCT/EP2014/077513

§ 371 Date: Jun. 10, 2016

(30) Foreign Application Priority Data
Dec. 13, 2013 (DK) PA201370773

(51) Int. Cl. C12N 15/79; C12N 15/90; C12N 9/22; C12N 15/10

(52) U.S. Cl. CPC C12N 15/79; C12N 15/102; C12N 15/902; C12N 9/22; C12Y 301/00; C12N 2800/80

(57) ABSTRACT

The invention relates to a method to select transformed cells. In particular, the present invention relates to the use of a nuclease engineered to inactivate selectable marker which confers cell resistance to a toxic compound. The present invention relates to methods of modifying genome of a cell, preferably an algal cell comprising the present selection step. The present invention also relates to specific engineered nucleases, polynucleotides, vectors encoding thereof, kits and isolated cells comprising said nuclease.

### FIELD OF THE INVENTION

The invention relates to a method to select transformed cells. In particular, the present invention relates to the use of a nuclease engineered to inactivate endogenous selectable marker which confers cell resistance or sensitivity to a toxic compound. The present invention relates to methods of modifying genome of a cell, preferably an algal cell comprising the present selection step. The present invention also relates to specific engineered nucleases, polynucleotides, vectors encoding thereof, kits and isolated cells comprising said nuclease.

### BACKGROUND OF THE INVENTION

Applications of algal products range from simple biomass production for food, feed and fuels to valuable products such as cosmetics, pharmaceuticals, pigments, sugar polymers and food supplements.

As a particular group of microalgae, diatoms are one of the most ecologically successful unicellular phytoplankton on the planet, being responsible for approximately 20% of global carbon fixation, representing a major participant in the marine food web. One of the major potential commercial or technological applications of diatoms is the capacity to accumulate abundant amounts of lipid suitable for conversion to liquid fuels. Because of their high potential to produce large quantities of lipids and good growth efficiencies, they are considered as one of the best classes of algae for renewable biofuel production. As a particular group of microalgae, diatoms are the only major group of eukaryotic phytoplankton with a diplontic life history, in which all vegetative cells are diploid and meiosis produces short-lived, haploid gametes, suggesting an ancestral selection for a life history dominated by a duplicated (diploid) genome.

Although the genomes of several algal species have now been sequenced, very few genetic tools to explore microalgal genetics are available at this time, which considerably limits the use of these organisms for various biotechnological applications. The diploid genome organization and the unknown sexual reproduction properties in these model species impede classical approaches based on random mutagenesis and phenotypic selection. The generation of strains with a modulated gene expression resides mainly on the use of random gene over-expression and targeted gene-silencing system using RNA interference (RNAi) (Siaut, Heijde et al. 2007; De Riso, Raniello et al. 2009).

Recently, the ability to perform targeted genomic manipulations within algal genome was facilitated by the use of homing endonuclease (WO 2012/017329).

Nevertheless, due to low transformation rates and the weak expression of transgenes, transformation methods require effective selection markers to discriminate successful transformed cells. However, only few publications refer to selection markers usable in Diatoms. Three antibiotics are shown to suppress the growth of cells and are used to select diatom transformed cells. (Dunahay, Jarvis et al. 1995; Zaslayskaia, Lippmeier et al. 2001) report the use of the neomycin phosphotransferase II (nptll), which inactivates G418 by phosphorylation, in Cyclotella cryptica, Navicula saprophila and Phaeodactylum tricornutum species. (Falciatore, Casotti et al. 1999; Zaslayskaia, Lippmeier et al. 2001) report the use of the Zeocin or Phleomycin resistance gene (Sh ble), acting by stochiometric binding, in Phaeodactylum tricornutum and Cylindrotheca fusiformis species. In (Zaslayskaia, Lippmeier et al. 2001), the use of N-acetyltransferase 1 gene (Nat1) conferring the resistance to Nourseothricin by enzymatic acetylation is reported in Phaeodactylum tricornutum and Thalassiosira pseudonana.

Moreover, public concern about widespread use of antibiotic resistance markers has prompted the inventor to develop an alternative marker system which consists to use nucleases for targeting genes for which their inactivation allows selection of transformed cells. This method offers two advantages, firstly the identification of new selectable marker for the diatoms for which only few antibiotic and herbicide markers are available and secondly the selection of transformed cells without any antibiotic gene integrated into the genome, thus allowing generation of no genetically modified organisms. This selection requires the inactivation of both alleles for diploid strain and only one for haploid strain and can be considered by the ability of the nucleases to induce high frequency of targeted mutagenesis.

### SUMMARY OF THE INVENTION

The inventor develops a selection method based on the inactivation of a gene which confers resistance to a toxic substrate. In particularly, the inventor proposes to start this proof of principle by inactivating the key enzyme in the synthesis of pyrimidines as uridine-5′-monophosphate synthase (UMPS), the nitrate reductase gene or the tryptophane synthetase. The inactivation of these genes has been shown to confer respectively the resistance to 5-Fluoroorotic acid (FOA) (Sakaguchi, Nakajima et al. 2011), chlorate (Daboussi, Djeballi et al. 1989) and 5-fluoroindole (Rohr, Sarkar et al. 2004; Falciatore, Merendino et al. 2005).

This method is particularly suitable for the selection of inactivated gene transformed cells by co-transformation of the nuclease targeting one of selectable marker genes with another protein of interest. The protein of interest can be a nuclease targeting a gene of interest to inactivate or a protein which increases the usability value of the algae in biotechnological applications. This co-transformation could be performed using multiple plasmids or using only one plasmid. Thus, we increase the proportion of transformed cells resistant to positive selection marker (5-FOA or Chlorate) containing the nuclease targeting the gene of interest. The delivery could be done by biolistic transformation, electroporation, micro-injection but also protein delivery using cell penetrating peptides, thus allowing the generation of no genetically modified organisms without transgene integration within the genome.

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The present invention relates to a selection method based on the inactivation of a selectable marker gene which confers resistance to a toxic substrate. This method comprises the step of introducing into a cell a nuclease capable of cleaving said selectable marker gene, and selecting cells resistant to said toxic compound. Particularly, the present invention relates to a method to select transformed cell comprising:
(a) Selecting a selectable marker gene within a genome of a cell which encodes a protein rendering a cell sensitive to a toxic substrate;
(b) Providing a nuclease which specifically recognizes and cleaves said selectable marker gene;
(c) Introducing said nuclease into a cell such that said nuclease cleavage inactivates said selectable gene;
(d) Culturing said cell with said toxic substrate and;
(e) Selecting cells which are resistant to the toxic substrate.

Selectable markers according to the present invention serve to eliminate unwanted elements. In particular, selectable marker gene is an endogenous gene which confers sensitivity to medium comprising a toxic substrate. Thus, inactivation of the selectable marker gene confers resistance to medium comprising toxic substrate. These markers are often toxic or otherwise inhibitory to replication under certain conditions. Consequently, it is possible to select cell comprising inactivated selectable marker gene. Selection of cells can also be obtained through the use of strains auxotropic for a particular metabolite. A point mutation or deletion in a gene required for amino acid synthesis or carbon source metabolism as non limiting examples can be used to select against strains when grown on media lacking the required nutrient. In most cases a defined “minimal” media is required for selection. There are a number of selective auxotropic markers that can be used in rich media, such as thyA and dapA-E from E. coli.

As non limiting examples, said selectable markers can be the tetAR gene which confers resistance to tetracycline but sensitivity to lipophilic component such as fusaric and quinalic acids (Bochner, Huang et al. 1980; Maloy and Nunn 1981), sacB b. subtilis gene encoding levansucrase that converts sucrose to levans which is harmful to the bacteria (Steinmetz, Le Coq et al. 1983; Gay, Le Coq et al. 1985), rpsL gene encoding the ribosomal subunit protein (S12) target of streptomycin (Dean 1981), ccdB encoding a cell-killing protein which is a potent poison of bacterial gyrase (Bernard, Gabant et al. 1994), PheS encoding the alpha subunits of the Phe-tRNA synthetase, which renders bacteria sensitive to p-chlorophenylalanine (Kast 1994), a phenylalanine analog, thya gene encoding a Thymidine synthetase which confers sensitivity to trimethoprim and related compounds (Stacey and Simson 1965), lacY encoding lactose permease, which renders bacteria sensitive to t-o-nitrophenyl-β-D-galactopyranoside (Murphy, Stewart et al. 1995), the amiE gene encoding a protein which converts fluoroacetamide to the toxic compound fluoroacetate (Collier, Spence et al. 2001), mazF gene, thymidine kinase, the Uridine 5′-monophosphate synthase gene (UMPS) encoding a protein which is involved in de novo synthesis of pyrimidine nucleotides and conversion of 5-Fluoroorotic acid (5-FOA) into the toxic compound 5-fluorouracil leading to cell death (Sakaguchi, Nakajima et al. 2011), the nitrate reductase gene encoding a protein which confers sensitivity to chlorate (Daboussi, Djeballi et al. 1989), the tryptophane synthase gene which converts the indole analog 5-fluoroindole (5-FI) into the toxic tryptophan analog 5-fluorotryptophan (Rohr, Sarkar et al. 2004; Falciatore, Merendino et al. 2005). According to the present invention, said selectable marker can be homologous sequences of the different genes described above. Here, homology between protein or DNA sequences is defined in terms of shared ancestry. Two segments of DNA can have shared ancestry because of either a speciation event (orthologs) or a duplication event (paralogs). In a preferred embodiment, said cell is an algal cell, more preferably a diatom and said selectable marker genes is UMPS or nitrate reductase gene.

Inactivation of these selectable marker genes confers sensitivity to a toxic substrate. By inactivating a gene it is intended that the gene of interest is not expressed in a functional protein form. In particular embodiment, the genetic modification of the method relies on the expression, in provided cells to engineer, of one nuclease such that said nuclease specifically catalyzes cleavage in one targeted gene thereby inactivating said targeted gene. The nucleic acid strand breaks caused by the nuclease are commonly repaired through the distinct mechanisms of homologous recombination or non-homologous end joining (NHEJ). However, NHEJ is an imperfect repair process that often results in changes to the DNA sequence at the site of the cleavage. Mechanisms involve rejoining of what remains of the two DNA ends through direct re-ligation (Critchlow and Jackson 1998) or via the so-called microhomology-mediated end joining (Ma, Kim et al. 2003). Repair via non-homologous end joining (NHEJ) often results in small insertions or deletions and can be used for the creation of specific gene knockouts. Said modification may be a substitution, deletion, or addition of at least one nucleotide.

Said nuclease can be a wild type or variant enzyme capable of catalyzing the hydrolysis (cleavage) of bonds between nucleic acids within a DNA or RNA molecule, preferably a DNA molecule. Particularly, said nuclease can be an endonuclease, more preferably a rare-cutting endonuclease which is highly specific, recognizing nucleic acid target sites ranging from 10 to 45 base pairs (bp) in length, usually ranging from 10 to 35 base pairs in length. The endonuclease according to the present invention recognizes and cleaves nucleic acid at specific polynucleotide sequences, further referred to as “target sequence”. The rare-cutting endonuclease can recognize and generate a single- or double-strand break at specific polynucleotides sequences.

In a particular embodiment, said rare-cutting endonuclease according to the present invention can be a Cas9 endonuclease. Indeed, recently a new genome engineering tool has been developed based on the RNA-guided Cas9 nuclease (Gasiunas, Barrangou et al. 2012; Jinek, Chylinski et al. 2012; Cong, Ran et al. 2013; Mali, Yang et al. 2013) from the type II prokaryotic CRISPR (Clustered Regularly Interspaced Short palindromic Repeats) adaptive immune system (see for review (Sorek, Lawrence et al. 2013)). The CRISPR Associated (Cas) system was first discovered in bacteria and functions as a defense against foreign DNA, either viral or plasmid. CRISPR-mediated genome engineering first proceeds by the selection of target sequence often flanked by a short sequence motif, referred as the proto-spacer adjacent motif (PAM). Following target sequence selection, a specific crRNA, complementary to this target sequence is engineered. Trans-activating crRNA (tracrRNA) required in the CRISPR type II systems paired to the crRNA and bound to the provided Cas9 protein. Cas9 acts as a molecular anchor facilitating the base pairing of tracRNA with cRNA (Deltcheva, Chylinski et al. 2011). In this ternary complex, the dual tracrRNA:crRNA structure acts as guide RNA that directs the endonuclease Cas9 to the cognate target sequence. Target recognition by the Cas9-tracrRNA:crRNA complex is initiated by scanning the target sequence for homology between the target sequence and the crRNA. In addition to the target sequence-crRNA complementarity, DNA targeting requires the presence of a short motif adjacent to the protospacer (protospacer adjacent motif—PAM). Following pairing between the dual-RNA and the target sequence, Cas9 subsequently introduces a blunt double strand break 3 bases upstream of the PAM motif (Garneau, Dupuis et al. 2010). In the present invention, guide RNA can be designed to specifically target said selectable marker. Following the pairing between the guide RNA and the target sequence, Cas9 induce a cleavage (double strand break or single strand break) within selectable marker gene. By Cas9 is also meant an engineered endonuclease or a homologue of Cas9 or split Cas9 which is capable of processing target nucleic acid sequence. By “Split Cas9” is meant here a reduced or truncated form of a Cas9 protein or Cas9 variant, which comprises either a RuvC or HNH domain, but not both of these domains. Such “Split Cas9” can be used independently with guide RNA or in a complementary fashion, like for instance, one Split Cas9 providing a RuvC domain and another providing the HNH domain. Different split Cas9 may be used together having either RuvC and/or NHN domains.

Rare-cutting endonuclease can also be a homing endonuclease, also known under the name of meganuclease. Such homing endonucleases are well-known to the art (Stoddard 2005). Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Preferred homing endonuclease according to the present invention can be an I-Crel variant. A “variant” endonuclease, i.e. an endonuclease that does not naturally exist in nature and that is obtained by genetic engineering or by random mutagenesis can bind DNA sequences different from that recognized by wild-type endonucleases (see international application WO2006/097854).

Said rare-cutting endonuclease can be a modular DNA binding nuclease. By modular DNA binding nuclease is meant any fusion proteins comprising at least one catalytic domain of an endonuclease and at least one DNA binding domain or protein specifying a nucleic acid target sequence. The DNA binding domain is generally a RNA or DNA-binding domain formed by an independently folded polypeptide protein domain that contains at least one motif that recognizes double- or single-stranded polynucleotides. Many such polypeptides have been described in the art having the ability to bind specific nucleic acid sequences. Such binding domains often comprise, as non limiting examples, helix-turn helix domains, leucine zipper domains, winged helix domains, helix-loop-helix domains, HMG-box domains, Immunoglobin domains, B3 domain or engineered zinc finger domain.

According to a preferred embodiment of the invention, the DNA binding domain is derived from a Transcription Activator like Effector (TALE), wherein sequence specificity is driven by a series of 33-35 amino acids repeats originating from Xanthomonas or Ralstonia bacterial proteins. These repeats differ essentially by two amino acids positions that specify an interaction with a base pair (Boch, Scholze et al. 2009; Moscou and Bogdanove 2009). Each base pair in the DNA target is contacted by a single repeat, with the specificity resulting from the two variant amino acids of the repeat (the so-called repeat variable dipeptide, RVD). TALE binding domains may further comprise an N-terminal translocation domain responsible for the requirement of a first thymine base (T₀) of the targeted sequence and a C-terminal domain that containing a nuclear localization signals (NLS). A TALE nucleic acid binding domain generally corresponds to an engineered core TALE scaffold comprising a plurality of TALE repeat sequences, each repeat comprising a RVD specific to each nucleotides base of a TALE recognition site. In the present invention, each TALE repeat sequence of said core scaffold is made of 30 to 42 amino acids, more preferably 33 or 34 wherein two critical amino acids (the so-called repeat variable dipeptide, RVD) located at positions 12 and 13 mediates the recognition of one nucleotide of said TALE binding site sequence; equivalent two critical amino acids can be located at positions other than 12 and 13 specially in TALE repeat sequence taller than 33 or 34 amino acids long. Preferably, RVDs associated with recognition of the different nucleotides are HD for recognizing C, NG for recognizing T, NI for recognizing A, NN for recognizing G or A. In another embodiment, critical amino acids 12 and 13 can be mutated towards other amino acid residues in order to modulate their specificity towards nucleotides A, T, C and G and in particular to enhance this specificity. A TALE nucleic acid binding domain usually comprises between 8 and 30 TALE repeat sequences. More preferably, said core scaffold of the present invention comprises between 8 and 20 TALE repeat sequences; again more preferably 15 TALE repeat sequences. It can also comprise an additional single truncated TALE repeat sequence made of 20 amino acids located at the C-terminus of said set of TALE repeat sequences, i.e. an additional C-terminal half-TALE repeat sequence.

Other engineered DNA binding domains are modular base-per-base specific nucleic acid binding domains (MBBBD) (PCT/US2013/051783). Said MBBBD can be engineered, for instance, from the newly identified proteins, namely EAV36_BURRH, E5AW43_BURRH, E5AW45_BURRH and E5AW46_BURRH proteins from the recently sequenced genome of the endosymbiont fungi Burkholderia Rhizoxinica (Lackner, Moebius et al. 2011). MBBBD proteins comprise modules of about 31 to 33 amino acids that are base specific. These modules display less than 40% sequence identity with Xanthomonas TALE common repeats, whereas they present more polypeptides sequence variability. When they are assembled together, these modular polypeptides can although target specific nucleic acid sequences in a quite similar fashion as Xanthomonas TALE-nucleases. According to a preferred embodiment of the present invention, said DNA binding domain is an engineered MBBBD binding domain comprising between 10 and 30 modules, preferably between 16 and 20 modules. The different domains from the above proteins (modules, N and C-terminals) from Burkholderia and Xanthomonas are useful to engineer new proteins or scaffolds having binding properties to specific nucleic acid sequences. In particular, additional N-terminal and C-terminal domains of engineered MBBBD can be derived from natural TALE like AvrBs3, PthXo1, AvrHah1, PthA, Tal1c as non-limiting examples.

“TALE-nuclease” or “MBBBD-nuclease” refers to engineered proteins resulting from the fusion of a DNA binding domain typically derived from Transcription Activator like Effector proteins (TALE) or MBBBD binding domain, with an endonuclease catalytic domain. Such catalytic domain is preferably a nuclease domain and more preferably a domain having endonuclease activity, like for instance I-Tevl, ColE7, NucA and Fok-I. In a particular embodiment, said nuclease is a monomeric TALE-Nuclease or MBBBD-nuclease. A monomeric Nuclease is a nuclease that does not require dimerization for specific recognition and cleavage, such as the fusions of engineered DNA binding domain with the catalytic domain of I-Tevl described in WO2012138927. In another particular embodiment, said rare-cutting endonuclease is a dimeric TALE-nuclease or MBBBD-nuclease, preferably comprising a DNA binding domain fused to Fokl. Said dimeric nuclease comprises a first DNA binding nuclease capable of binding a target sequence comprising a part of the repeat sequence and a sequence adjacent thereto and a second DNA binding nuclease capable of binding a target sequence within the repeat sequence, such that the dimeric nuclease induces a cleavage event within the repeat sequence. TALE-nuclease have been already described and used to stimulate gene targeting and gene modifications (Boch, Scholze et al. 2009; Moscou and Bogdanove 2009; Cermak, Doyle et al. 2010; Christian, Cermak et al. 2010). Such engineered TALE-nucleases are commercially available under the trade name TALEN™ (Cellectis, 8 rue de la Croix Jarry, 75013 Paris, France).

In another embodiment, additional catalytic domain can be further introduced into the cell with said nuclease to increase mutagenesis in order to enhance their capacity to inactivate targeted genes. In particular, said additional catalytic domain is a DNA end processing enzyme. Non limiting examples of DNA end-processing enzymes include 5-3′ exonucleases, 3-5′ exonucleases, 5-3′ alkaline exonucleases, 5′ flap endonucleases, helicases, hosphatase, hydrolases and template-independent DNA polymerases. Non limiting examples of such catalytic domain comprise of a protein domain or catalytically active derivate of the protein domain selected from the group consisting of hExol (EXO1_HUMAN), Yeast Exol (EXO1_YEAST), E.coli Exol, Human TREX2, Mouse TREX1, Human TREX1, Bovine TREX1, sae2 nuclease (CtBP-intracting protein (CtIP) homologue), Rat TREX1, TdT (terminal deoxynucleotidyl transferase) Human DNA2, Yeast DNA2 (DNA2_YEAST). In a preferred embodiment, said additional catalytic domain has a 3′-5′-exonuclease activity, and in a more preferred embodiment, said additional catalytic domain is TREX, more preferably TREX2 catalytic domain (WO2012/058458). In another preferred embodiment, said catalytic domain is encoded by a single chain TREX2 polypeptide. Said additional catalytic domain may be fused to a nuclease fusion protein or chimeric protein according to the invention optionally by a peptide linker.

Endonucleolytic breaks are known to stimulate the rate of homologous recombination. Thus, in another embodiment, the genetic modification step of the method further comprises a step of introduction into cells a donor matrix comprising at least a sequence homologous to a portion of the target nucleic acid sequence, such as the selectable marker gene, such that homologous recombination occurs between the target nucleic acid sequence and the donor matrix. In particular embodiments, said donor matrix comprises first and second portions which are homologous to region 5′ and 3′ of the target nucleic acid sequence, respectively. Preferably, homologous equences of at least 50 bp, preferably more than 100 bp and more preferably more than 200 bp are used within said donor matrix. Therefore, the homologous sequence is preferably from 200 bp to 6000 bp, more preferably from 1000 bp to 2000 bp. Indeed, shared nucleic acid homologies are located in regions flanking upstream and downstream the site of the break and the nucleic acid sequence to be introduced should be located between the two arms.

In particular, said donor matrix successively comprises a first region of homology to sequences upstream of said cleavage, a sequence to inactivate one selectable marker gene and a second region of homology to sequences downstream of the cleavage. Said polynucleotide introduction step can be simultaneous, before or after the introduction or expression of said nuclease. Depending on the location of the target nucleic acid sequence wherein break event has occurred, such donor matrix can be used to knock-out a gene, e.g. when exogenous nucleic acid is located within the open reading frame of said gene, or to introduce new sequences or genes of interest. New sequences or gene of interest can encode a protein of interest, preferably a protein which increases the potential exploitation of algae by conferring them commercially desirable trait for various biotechnological applications or a nuclease which specifically targets a gene to inactivate within cell genome. Sequence insertions by using such donor matrix can be used to modify a targeted existing gene, by correction or replacement of said gene (allele swap as a non-limiting example), or to up- or down-regulate the expression of the targeted gene (promoter swap as non-limiting example), said targeted gene correction or replacement.

The method of the present invention can further comprise introducing another protein of interest into a cell. Preferably, the protein of interest is useful for increasing the usability and the commercial value of algae for various biotechnological applications. In a more preferred embodiment, said protein is involved in the lipid metablolism. The protein of interest can also be a nuclease which can recognize and cleave a target sequence of interest. Resulting gene inactivation can increase the potential exploitation of algae by conferring them commercially desirable traits for various biotechnological applications, such as biofuel production.

In a more preferred embodiment said protein of interest can be introduced as a transgene into the cell. Said transgenes encoding said protein of interest and nuclease cleaving selectable marker gene according to the present invention can be encoded by one or as different nucleic acid, preferably different vectors. Different transgenes can be included in one vector which comprises a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82: 1013-1025 (2001); Donnelly et al., J. of Gen. Virology 78: 13-21 (1997); Doronina et al., Mol. And. Cell. Biology 28(13): 4227-4239 (2008); Atkins et al., RNA 13: 803-810 (2007)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA. As non-limiting example, in the present invention, 2A peptides have been used to express into the cell the nuclease cleaving the selectable marker gene, and the nuclease cleaving the gene of interest to inactivate, the DNA end-processing enzyme, the donor matrix or another transgene encoding a protein of interest.

Delivery Method

A variety of different methods are known for introducing protein of interest into cells. In various embodiments, said nuclease cleaving the selectable marker gene or other protein of interest can be encoded by a transgene, preferably comprised within a vector. In another embodiment, said protein of interest is encoded by RNA sequence. Said vectors or RNA sequence can be introduced into cell by, for example without limitation, electroporation, magnetophoresis. The latter is a nucleic acid introduction technology using the processes of magnetophoresis and nanotechnology fabrication of micro-sized linear magnets (Kuehnle et al., U.S. Pat. No. 6,706,394; 2004; Kuehnle et al., U.S. Pat. No. 5,516,670; 1996) that proved amenable to effective chloroplast engineering in freshwater Chlamydomonas, improving plastid transformation efficiency by two orders of magnitude over the state-of the-art of biolistics (Champagne et al., Magnetophoresis for pathway engineering in green cells. Metabolic engineering V: Genome to Product, Engineering Conferences International Lake Tahoe Calif., Abstracts pp 76; 2004). Polyethylene glycol treatment of protoplasts is another technique that can be used to transform cells (Maliga 2004). In various embodiments, the transformation methods can be coupled with one or more methods for visualization or quantification of nucleic acid introduction into cell. Also appropriate mixtures commercially available for protein transfection can be used to introduce protein in algae. More broadly, any means known in the art to allow delivery inside cells or subcellular compartments of agents/chemicals and molecules (proteins) can be used including liposomal delivery means, polymeric carriers, chemical carriers, lipoplexes, polyplexes, dendrimers, nanoparticles, emulsion, natural endocytosis or phagocytose pathway as non-limiting examples. Direct introduction, such as microinjection of protein of interest or DNA in cell can be considered. In a more preferred embodiment, said transformation construct is introduced into host cell by particle inflow gun bombardment or electroporation.

In another particular embodiment, said transgene or protein of interest can be introduced into the cell by using cell penetrating peptides (CPP). Said CPP can be associated with the transgene or protein of interest (named cargo molecule). This association can be covalent or non-covalent. CPPs can be subdivided into two main classes, the first requiring chemical linkage with the cargo and the second involving the formation of stable, non-covalent complexes. Said cargo molecule can be as non limiting example polynucleotides of either the DNA or RNA type, preferably polynucleotides encoding protein of interest, such as nuclease, marker molecule, proteins of interest useful to engineer the genetics of the algae, in particular, proteins involved in fatty acid metabolism, carbohydrate metabolism, genes associated with stress tolerance in growth conditions, and the like. Said cargo molecules can also be genes, expression cassettes, plasmids, sRNA, siRNA, miRNA shRNA, guide RNA of the CRISPR system and polypeptides such as protein of interest, nuclease, marker molecule.

Although definition of CPPs is constantly evolving, they are generally described as short peptides of less than 35 amino acids either derived from proteins or from chimeric sequences which are capable of transporting polar hydrophilic biomolecules across cell membrane in a receptor independent manner. CPP can be cationic peptides, peptides having hydrophobic sequences, amphipatic peptides, peptides having proline-rich and anti-microbial sequence, and chimeric or bipartite peptides (Pooga and Langel 2005). In a particular embodiment, cationic CPP can comprise multiple basic of cationic CPPs (e.g., arginine and/or lysine). Preferably, CCP are amphipathic and possess a net positive charge. CPPs are able to penetrate biological membranes, to trigger the movement of various biomolecules across cell membranes into the cytoplasm and to improve their intracellular routing, thereby facilitating interactions with the target. Examples of CPP can include as non limiting examples: Tat, a nuclear transcriptional activator protein which is a 101 amino acid protein required for viral replication by human immunodeficiency virus type 1 (HIV-1), penetratin, which corresponds to the third helix of the homeoprotein Antennapedia in Drosophilia, Kaposi fibroblast growth factor (FGF) signal peptide sequence, integrin β3 signal peptide sequence; MPG; pep-1; sweet arrow peptide, dermaseptins, transportan, pVEC, Human calcitonin, mouse prion protein (mPrPr) (REF: US2013/0065314).

TALE-Nucleases

In another aspect, the present invention also relates to the nuclease disclosed here. In particular embodiment, the present invention relates to a nuclease capable of recognizing a target sequence within the UMPS gene or the nitrate reductase gene, preferably within the P. tricornutum UMPS gene (SEQ ID NO:1, GenBank: AB512669.1) or the P. tricornutum nitrate reductase gene (SEQ ID NO: 2, GenBank: AY579336.1), in a preferred embodiment a target sequence within the UMPS or nitrate reductase gene having at least 70%, preferably 80%, 85%, 90%; 95% identity with the nucleic acid sequence SEQ ID NO: 1 or 2. In a particular embodiment, said target sequence is selected from the group consisting of: SEQ ID NO: 3 and SEQ ID NO: 4, in a preferred embodiment, said target sequence has at least 70%, preferably 80%, 85%, 90%; 95% identity with the nucleic acid sequence selected from the group consisting of: SEQ ID NO: 3 and SEQ ID NO: 4.

In a particular embodiment, said nuclease is a TALE-nuclease. In a more particular embodiment, the present invention relates to a TALE-nuclease having amino acid sequence selected from the group consisting of: SEQ ID NO: 5 to SEQ ID NO: 8. In a preferred embodidment said TALE-nuclease has at least 70%, preferably 80%, 85%, 90%; 95% identity with the amino acid sequence selected from the group consisting of: SEQ ID NO: 5 to SEQ ID NO: 8.

Polynucleotides, Vectors

The invention also concerns the polynucleotides, in particular DNA or RNA encoding the nucleases previously described. These polynucleotides may be included in vectors, more particularly plasmids or virus, in view of being expressed in prokaryotic or eukaryotic cells. The polynucleotide may consist in an expression cassette or expression vector (e.g. a plasmid for introduction into a bacterial host cell, or a viral vector such as a baculovirus vector for transfection of an insect host cell, or a plasmid or viral vector such as a lentivirus for transfection of a mammalian host cell). In a particular embodiment, the present invention relates to a polynucleotide comprising the nucleic acid sequence SEQ ID NO: 9 to SEQ ID NO: 12. Those skilled in the art will recognize that, in view of the degeneracy of the genetic code, considerable sequence variation is possible among these polynucleotide molecules. Preferably, the nucleic acid sequences of the present invention are codon-optimized for expression in algal cells, preferably for expression in diatom cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged. In a preferred embodiment, the polynucleotide has at least 70%, preferably at least 80%, more preferably at least 90%, 95% 97% or 99% sequence identity with nucleic acid sequence selected from the group consisting of SEQ ID NO: 9 to SEQ ID NO: 12.

Isolated Cells

In another aspect, the present invention relates to an isolated cell obtainable or obtained by the method described above. In particular, the present invention relates to a cell, preferably an algal cell which comprises a nuclease capable of recognizing and cleaving a selectable marker gene, preferably a UMPS or nitrate reductase gene. In the frame of the present invention, “algae” or “algae cells” refer to different species of algae that can be used as host for selection method using nuclease of the present invention. Algae are mainly photoautotrophs unified primarily by their lack of roots, leaves and other organs that characterize higher plants. Term “algae” groups, without limitation, several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from: Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

In a more preferred embodiment, algae are diatoms. Diatoms are unicellular phototrophs identified by their species-specific morphology of their amorphous silica cell wall, which vary from each other at the nanometer scale. Diatoms includes as non limiting examples: Phaeodactylum, Fragilariopsis, Thalassiosira, Coscinodiscus, Arachnoidiscusm, Aster omphalus, Navicula, Chaetoceros, Chorethron, Cylindrotheca fusiformis, Cyclotella, Lampriscus, Gyrosigma, Achnanthes, Cocconeis, Nitzschia, Amphora, schizochytrium and Odontella. In a more preferred embodiment, diatoms according to the invention are from the species: Thalassiosira pseudonana or Phaeodactylum tricornutum.

Kits

Another aspect of the invention is a kit for algal cell selection comprising a nuclease which recognizes and cleaves a selectable marker as previously described. This kit more particularly comprises a nuclease capable of recognizing and cleaving a UMPS or nitrate reductase gene, optionally with the adequate toxic substrate for cell selection, such as chlorate or 5′FOA. In particular, the kit may comprise a TALE-nuclease having at least 70%, preferably at least 80%, more preferably at least 90%, 95% 97% or 99% sequence identity with amino acid sequence sequence selected from the group consisting of SEQ ID NO: 5 to SEQ ID NO: 8. The kit may further comprise one or several components required to realize the selection method as described above.

Definitions

In the description above, a number of terms are used extensively. The following definitions are provided to facilitate understanding of the present embodiments.

As used herein, “a” or “an” may mean one or more than one.
Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
As used herein, “nucleic acid” or “nucleic acid molecule” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Nucleic acids can be either single stranded or double stranded.
By “gene” is meant the basic unit of heredity, consisting of a segment of DNA arranged in a linear manner along a chromosome, which codes for a specific protein or segment of protein. A gene typically includes a promoter, a 5′ untranslated region, one or more coding sequences (exons), optionally introns, a 3′ untranslated region. The gene may further comprise a terminator, enhancers and/or silencers.

By “genome” it is meant the entire genetic material contained in a cell such as nuclear genome, chloroplastic genome, mitochondrial genome.
By “target sequence” is intended a polynucleotide sequence that can be processed by a rare-cutting endonuclease according to the present invention. These terms refer to a specific DNA location, preferably a genomic location in a cell, but also a portion of genetic material that can exist independently to the main body of genetic material such as plasmids, episomes, virus, transposons or in organelles such as mitochondria or chloroplasts as non-limiting examples. The nucleic acid target sequence is defined by the 5′ to 3′ sequence of one strand of said target.
As used herein, the term transgene means a nucleic acid sequence (encoding, e.g., one or more polypeptides), which is partly or entirely heterologous, i.e., foreign, to the host cell into which it is introduced, or, is homologous to an endogenous gene of the host cell into which it is introduced, but which can be designed to be inserted, or can be inserted, into the cell genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of the selected nucleic acid encoding polypeptide. The polypeptide encoded by the transgene can be either not expressed, or expressed but not biologically active, in the algae or algal cells in which the transgene is inserted. Also, the transgene can be a sequence inserted in the genome for producing an interfering RNA. Most preferably, the transgene encodes a polypeptide useful for increasing the quantity and/or the quality of the lipid in the diatom.
By “homologous” it is meant a sequence with enough identity to another one to lead to homologous recombination between sequences, more particularly having at least 95% identity, preferably 97% identity and more preferably 99%.
“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.
By “vector” is intended to mean a nucleic acid molecule capable of transporting another nucleic acid to which it has been linked. A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those skilled in the art and commercially available. Some useful vectors include, for example without limitation, pGEM13z. pGEMT and pGEMTEasy {Promega, Madison, Wis.); pSTBluel (EMD Chemicals Inc. San Diego, Calif.); and pcDNA3.1, pCR4-TOPO, pCR-TOPO-II, pCRBlunt-II-TOPO (Invitrogen, Carlsbad, Calif.). Preferably said vectors are expression vectors, wherein the sequence(s) encoding the rare-cutting endonuclease of the invention is placed under control of appropriate transcriptional and translational control elements to permit production or synthesis of said rare-cutting endonuclease. Therefore, said polynucleotide is comprised in an expression cassette. More particularly, the vector comprises a replication origin, a promoter operatively linked to said polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said rare-cutting endonuclease is a heterodimer, the two polynucleotides encoding each of the monomers are included in two vectors to avoid intraplasmidic recombination events. In another embodiment the two polynucleotides encoding each of the monomers are included in one vector which is able to drive the expression of both polynucleotides, simultaneously. In some embodiments, the vector for the expression of the rare-cutting endonucleases according to the invention can be operably linked to an algal-specific promoter. In some embodiments, the algal-specific promoter is an inducible promoter. In some embodiments, the algal-specific promoter is a constitutive promoter. Promoters that can be used include, for example without limitation, a Pptca1 promoter (the CO2 responsive promoter of the chloroplastic carbonic anyhydrase gene, ptcal, from P. tricornutum), a NITI promoter, an AMTI promoter, an AMT2 promoter, an AMT4 promoter, a RHI promoter, a cauliflower mosaic virus 35S promoter, a tobacco mosaic virus promoter, a simian virus 40 promoter, a ubiquitin promoter, a PBCV-I VP54 promoter, or functional fragments thereof, or any other suitable promoter sequence known to those skilled in the art. In another more preferred embodiment according to the present invention the vector is a shuttle vector, which can both propagate in E. coli (the construct containing an appropriate selectable marker and origin of replication) and be compatible for propagation or integration in the genome of the selected algae.
The term “promoter” as used herein refers to a minimal nucleic acid sequence sufficient to direct transcription of a nucleic acid sequence to which it is operably linked. The term “promoter” is also meant to encompass those promoter elements sufficient for promoter-dependent gene expression controllable for cell-type specific expression, tissue specific expression, or inducible by external signals or agents; such elements may be located in the 5′ or 3′ regions of the naturally-occurring gene.

By “inducible promoter” it is mean a promoter that is transcriptionally active when bound to a transcriptional activator, which in turn is activated under a specific condition(s), e.g., in the presence of a particular chemical signal or combination of chemical signals that affect binding of the transcriptional activator, e.g., CO₂ or NO₂, to the inducible promoter and/or affect function of the transcriptional activator itself.

The term “host cell” refers to a cell that is transformed using the methods of the invention. In general, host cell as used herein means an algal cell into which a nucleic acid target sequence has been modified.

By “mutagenesis” is understood the elimination or addition of at least one given DNA fragment (at least one nucleotide) or sequence, bordering the recognition sites of rare-cutting endonuclease.

By “NHEJ” (non-homologous end joining) is intended a pathway that repairs double-strand breaks in DNA in which the break ends are ligated directly without the need for a homologous template. NHEJ comprises at least two different processes. Mechanisms involve rejoining of what remains of the two DNA ends through direct re-ligation {Critchlow, 1998 #17} or via the so-called microhomology-mediated end joining (Akopian, He et al. 2003) that results in small insertions or deletions and can be used for the creation of specific gene knockouts.

The term “Homologous recombination” refers to the conserved DNA maintenance pathway involved in the repair of DSBs and other DNA lesions. In gene targeting experiments, the exchange of genetic information is promoted between an endogenous chromosomal sequence and an exogenous DNA construct. Depending of the design of the targeted construct, genes could be knocked out, knocked in, replaced, corrected or mutated, in a rational, precise and efficient manner. The process requires homology between the targeting construct and the targeted locus. Preferably, homologous recombination is performed using two flanking sequences having identity with the endogenous sequence in order to make more precise integration as described in WO9011354.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of”, “chosen from” and the like include mixtures of the specified materials.

Where a numerical limit or range is stated herein, the endpoints are included. Also, all values and sub-ranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1

### New Method for Selection of Diatom-Transformed Cells Using a TALEN Targeting the UMPS Gene and Conferring a Resistance to 5-FOA

Due to the very low transformation efficacy 10⁻⁸, the delivery of a protein encoding plasmid in the marine diatom Phaeodactylum tricornutum is mediated by co-transformation with antibiotic selectable marker.

Here, we propose the use of new selectable method which consists to the co-transformation of a plasmid encoding the protein of interest and plasmids encoding a TALEN targeting the gene UMPS encoding the key enzyme in the synthesis of pyrimidines: Uridine-5′-monophosphate synthase. The mutagenic events induced by this TALEN could lead to gene inactivation which has been previously reported to confer a resistance to 5-Fluoroorotic acid (5-FOA) as it has been previously reported (Sakaguchi, Nakajima et al. 2011). For that, a UMPS_TALEN (SEQ ID NO: 5 and SEQ ID NO: 6) encoded by the pCLS20603 (SEQ ID NO: 13) and pCLS20604 (SEQ ID NO: 14) plasmids designed to cleave the DNA sequence 5′-TTTAGTCTGTCTCTAGGTGTTCTCAAATTCGGCTCTTTTGTGCTGAAAA-3′ (SEQ ID NO: 3) were used. The diatoms transformed by this TALEN will be selected on 5-FOA medium according with the conditions described in (Sakaguchi, Nakajima et al. 2011).

Materials and Methods

Culture Conditions

Phaeodactylum tricornutum Bohlin clone CCMP2561 was grown in filtered Guillard's f/2 medium without silica (40°/°° w/v Sigma Sea Salts 59883, supplemented with 1× Guillard's f/2 marine water enrichment solution (Sigma G0154) in a Sanyo incubator (model MLR-351) at a constant temperature (20+/−0.5° C.). The incubator is equipped with white cold neon light tubes that produce an illumination of about 120 μmol photons m⁻² s⁻¹ and a photoperiod of 12 h light:12 h darkness (illumination period from 9 AM to 9 PM).

Genetic Transformation

5·10⁷ cells were collected from exponentially growing liquid cultures (concentration about 10⁶ cells/ml) by centrifugation (3000 rpm for 10 minutes at 20° C.). The supernatant was discarded and the cell pellet resuspended in 500 μl of fresh f/2 medium. The cell suspension was then spread on the center one-third of a 10 cm 1% agar plate containing 20°/°° sea salts supplemented with f/2 solution without silica. Two hours later, transformation was carried out using the microparticle bombardment (Biolistic PDS-1000/He Particle Delivery System (BioRad)). The protocol is adapted from (Falciatore, Casotti et al. 1999) and (Apt, Kroth-Pancic et al. 1996) with minor modifications. Briefly, M17 tungstene particles (1.1 μm diameter, BioRad) were coated with 6 μg of total amount of DNA containing 3 μg of each monomer of TALENs (pCLS20603 and pCLS20604), using 1.25M CaCl2 and 20 mM spermidine according to the manufacturer's instructions. As negative control, beads were coated with a DNA mixture containing 6 μg empty vector (pCLS0003) (SEQ ID NO: 17). Agar plates with the diatoms to be transformed were positioned at 7.5 cm from the stopping screen within the bombardment chamber (target shelf on position two). A burst pressure of 1550 psi and a vacuum of 25 Hg/in were used. After bombardment, plates were incubated for 48 hours with a 12 h light:12 h dark photoperiod.

Selection

Two days post transformation, bombarded cells were gently scrapped with 700 μl of f/2 medium without silica and spread on two 10 cm 1% agar plates (20°/°° sea salts supplemented with f/2 medium without silica) containing 5-FOA. Plates were then placed in the incubator under a 12 h light:12 h darkness cycle for at least three weeks.

Characterization

Resistant colonies were picked and dissociated in 20 μl of lysis buffer (1% TritonX-100, 20 mM Tris-HCl pH8, 2 mM EDTA) in an eppendorf tube. Tubes were vortexed for at least 30 sec and then kept on ice for 15 min. After heating for 10 min at 85° C., tubes were cooled down at RT and briefly centrifuged to pellet cells debris. Supernatants were used immediately or stocked at 4° C. 5 μl of a 1:5 dilution in milliQ H2O of the supernatants, were used for each PCR reaction. The UMPS target will be amplified using a 1:5 dilution of the lysis colony with specific primers and sequenced to identify the nature of mutagenic event.

Results

The transformation of diatoms with plasmids encoding TALEN would lead to the UMPS gene inactivation conferring the ability to grow on medium supplemented with 5-FOA.

### Example 2

### New Method for Selection of Diatom-Transformed Cells Using a TALEN Targeting the Nitrate Reductase Gene and Conferring a Resistance to Chlorate

Due to the very low transformation efficacy 10⁻⁸, the delivery of a protein encoding plasmid in the marine diatom Phaeodactylum tricornutum is mediated by co-transformation with antibiotic selectable marker.

Here, we propose the use of new selectable method which consists to the co-transformation of a plasmid encoding the protein of interest and plasmids encoding a TALEN targeting the gene NR encoding one key enzyme in the Nitrate metabolism: Nitrate reductase. The mutagenic events induced by this TALEN could lead to gene inactivation which has been previously reported to confer a resistance to Chlorate as it has been previously reported (Daboussi, Djeballi et al. 1989). For that, a NR_TALEN (SEQ ID NO: 7 and SEQ ID NO: 8) encoded by the pCLS16353 (SEQ ID NO: 15) and pCLS16354 (SEQ ID NO: 16) plasmids designed to cleave the DNA sequence 5′-TGAAGCAGCATCGATTTATTACGCCGTCCTCGTTGCATTACGTACGCAA-3′ (SEQ ID NO: 2) were used. The diatoms transformed by this TALEN will be selected on chlorate medium according with the conditions described in (Daboussi, Djeballi et al. 1989).

Materials and Methods

Culture Conditions

Phaeodactylum tricornutum Bohlin clone CCMP2561 was grown in filtered Guillard's f/2 medium without silica (40°/°° w/v Sigma Sea Salts 59883, supplemented with 1× Guillard's f/2 marine water enrichment solution (Sigma G0154) in a Sanyo incubator (model MLR-351) at a constant temperature (20+/−0.5° C.). The incubator is equipped with white cold neon light tubes that produce an illumination of about 120 μmol photons m⁻² s⁻¹ and a photoperiod of 12 h light:12 h darkness (illumination period from 9 AM to 9 PM).

Genetic Transformation

5·10⁷ cells were collected from exponentially growing liquid cultures (concentration about 10⁶ cells/ml) by centrifugation (3000 rpm for 10 minutes at 20° C.). The supernatant was discarded and the cell pellet resuspended in 500 μl of fresh f/2 medium. The cell suspension was then spread on the center one-third of a 10 cm 1% agar plate containing 20°/°° sea salts supplemented with f/2 solution without silica. Two hours later, transformation was carried out using the microparticle bombardment (Biolistic PDS-1000/He Particle Delivery System (BioRad)). The protocol is adapted from (Falciatore, Casotti et al. 1999) and (Apt, Kroth-Pancic et al. 1996) with minor modifications. Briefly, M17 tungstene particles (1.1 μm diameter, BioRad) were coated with 6 μg of total amount of DNA containing 3 μg of each monomer of TALENs (pCLS16353 and pCLS16354), using 1.25M CaCl2 and 20 mM spermidine according to the manufacturer's instructions. As negative control, beads were coated with a DNA mixture containing 6 μg empty vector (pCLS0003) (SEQ ID NO: 17). Agar plates with the diatoms to be transformed were positioned at 7.5 cm from the stopping screen within the bombardment chamber (target shelf on position two). A burst pressure of 1550 psi and a vacuum of 25 Hg/in were used. After bombardment, plates were incubated for 48 hours with a 12 h light:12 h dark photoperiod.

Selection

Two days post transformation, bombarded cells were gently scrapped with 700 μl of f/2 medium without silica and spread on two 10 cm 1% agar plates (20°/°° sea salts supplemented with f/2 medium without silica) containing Chlorate. Plates were then placed in the incubator under a 12 h light:12 h darkness cycle for at least three weeks.

Characterization

Resistant colonies were picked and dissociated in 20 μl of lysis buffer (1% TritonX-100, 20 mM Tris-HCl pH8, 2 mM EDTA) in an eppendorf tube. Tubes were vortexed for at least 30 sec and then kept on ice for 15 min. After heating for 10 min at 85° C., tubes were cooled down at RT and briefly centrifuged to pellet cells debris. Supernatants were used immediately or stocked at 4° C. 5 μl of a 1:5 dilution in milliQ H2O of the supernatants, were used for each PCR reaction. The UMPS target will be amplified using a 1:5 dilution of the lysis colony with specific primers and sequenced to identify the nature of mutagenic event.

Results

The transformation of diatoms with plasmids encoding TALEN would lead to the NR gene inactivation conferring the ability to grow on medium supplemented with chlorate.

### REFERENCES

Akopian, A., J. He, et al. (2003). “Chimeric recombinases with designed DNA sequence recognition.” Proc Natl Acad Sci USA 100(15): 8688-91.

Apt, K. E., P. G. Kroth-Pancic, et al. (1996). “Stable nuclear transformation of the diatom Phaeodactylum tricornutum.” Mol Gen Genet 252(5): 572-9.

Bernard, P., P. Gabant, et al. (1994). “Positive-selection vectors using the F plasmid ccdB killer gene.” Gene 148(1): 71-4.

Boch, J., H. Scholze, et al. (2009). “Breaking the code of DNA binding specificity of TAL-type III effectors.” Science 326(5959): 1509-12.

Bochner, B. R., H. C. Huang, et al. (1980). “Positive selection for loss of tetracycline resistance.” J Bacteriol 143(2): 926-33.

Cermak, T., E. L. Doyle, et al. (2010). “Efficient design and assembly of custom TALEN and other TAL effector-based constructs for DNA targeting.” Nucleic Acids Res 39(12): e82.

Christian, M., T. Cermak, et al. (2010). “Targeting DNA double-strand breaks with TAL effector nucleases.” Genetics 186(2): 757-61.

Collier, D. N., C. Spence, et al. (2001). “Isolation and phenotypic characterization of Pseudomonas aeruginosa pseudorevertants containing suppressors of the catabolite repression control-defective crc-10 allele.” FEMS Microbiol Lett 196(2): 87-92.

Cong, L., F. A. Ran, et al. (2013). “Multiplex genome engineering using CRISPR/Cas systems.” Science 339(6121): 819-23.

Critchlow, S. E. and S. P. Jackson (1998). “DNA end-joining: from yeast to man.” Trends Biochem Sci 23(10): 394-8.

Daboussi, M. J., A. Djeballi, et al. (1989). “Transformation of seven species of filamentous fungi using the nitrate reductase gene of Aspergillus nidulans.” Curr Genet 15(6): 453-6.

De Riso, V., R. Raniello, et al. (2009). “Gene silencing in the marine diatom Phaeodactylum tricornutum.” Nucleic Acids Res 37(14): e96.

Dean, D. (1981). “A plasmid cloning vector for the direct selection of strains carrying recombinant plasmids.” Gene 15(1): 99-102.

Deltcheva, E., K. Chylinski, et al. (2011). “CRISPR RNA maturation by trans-encoded small RNA and host factor RNase III.” Nature 471(7340): 602-7.

Dunahay, T. G., E. E. Jarvis, et al. (1995). “Genetic transformation of the diatoms Cyclotella Cryptica and Navicula Saprophila.” Journal of Phycology 31(6): 1004-1012.

Falciatore, A., R. Casotti, et al. (1999). “Transformation of Nonselectable Reporter Genes in Marine Diatoms.” Mar Biotechnol (NY) 1(3): 239-251.

Falciatore, A., L. Merendino, et al. (2005). “The FLP proteins act as regulators of chlorophyll synthesis in response to light and plastid signals in Chlamydomonas.” Genes Dev 19(1): 176-87.

Garneau, J. E., M. E. Dupuis, et al. (2010). “The CRISPR/Cas bacterial immune system cleaves bacteriophage and plasmid DNA.” Nature 468(7320): 67-71.

Gasiunas, G., R. Barrangou, et al. (2012). “Cas9-crRNA ribonucleoprotein complex mediates specific DNA cleavage for adaptive immunity in bacteria.” Proc Natl Acad Sci USA 109(39): E2579-86.

Gay, P., D. Le Coq, et al. (1985). “Positive selection procedure for entrapment of insertion sequence elements in gram-negative bacteria.” J Bacteriol 164(2): 918-21.

Jinek, M., K. Chylinski, et al. (2012). “A programmable dual-RNA-guided DNA endonuclease in adaptive bacterial immunity.” Science 337(6096): 816-21.

Kast, P. (1994). “pKSS—a second-generation general purpose cloning vector for efficient positive selection of recombinant clones.” Gene 138(1-2): 109-14.

Lackner, G., N. Moebius, et al. (2011). “Complete genome sequence of Burkholderia rhizoxinica, an Endosymbiont of Rhizopus microsporus.” J Bacteriol 193(3): 783-4.

Ma, J. L., E. M. Kim, et al. (2003). “Yeast Mre11 and Rad1 proteins define a Ku-independent mechanism to repair double-strand breaks lacking overlapping end sequences.” Mol Cell Biol 23(23): 8820-8.

Mali, P., L. Yang, et al. (2013). “RNA-guided human genome engineering via Cas9.” Science 339(6121): 823-6.

Maliga, P. (2004). “Plastid transformation in higher plants.” Annu Rev Plant Biol 55: 289-313.

Maloy, S. R. and W. D. Nunn (1981). “Selection for loss of tetracycline resistance by Escherichia coli.” J Bacteriol 145(2): 1110-1.

Moscou, M. J. and A. J. Bogdanove (2009). “A simple cipher governs DNA recognition by TAL effectors.” Science 326(5959): 1501.

Murphy, C. K., E. J. Stewart, et al. (1995). “A double counter-selection system for the study of null alleles of essential genes in Escherichia coli.” Gene 155(1): 1-7.

Pooga, M. and U. Langel (2005). “Synthesis of cell-penetrating peptides for cargo delivery.” Methods Mol Biol 298: 77-89.

Rohr, J., N. Sarkar, et al. (2004). “Tandem inverted repeat system for selection of effective transgenic RNAi strains in Chlamydomonas.” Plant J 40(4): 611-21.

Sakaguchi, T., K. Nakajima, et al. (2011). “Identification of the UMP synthase gene by establishment of uracil auxotrophic mutants and the phenotypic complementation system in the marine diatom Phaeodactylum tricornutum.” Plant Physiol 156(1): 78-89.

Siaut, M., M. Heijde, et al. (2007). “Molecular toolbox for studying diatom biology in Phaeodactylum tricornutum.” Gene 406(1-2): 23-35.

Sorek, R., C. M. Lawrence, et al. (2013). “CRISPR-mediated Adaptive Immune Systems in Bacteria and Archaea.” Annu Rev Biochem.

Stacey, K. A. and E. Simson (1965). “Improved Method for the Isolation of Thymine-Requiring Mutants of Escherichia Coli.” J Bacteriol 90: 554-5.

Steinmetz, M., D. Le Coq, et al. (1983). “[Genetic analysis of sacB, the structural gene of a secreted enzyme, levansucrase of Bacillus subtilis Marburg].” Mol Gen Genet 191(1): 138-44.

Stoddard, B. L. (2005). “Homing endonuclease structure and function.” Q Rev Biophys 38(1): 49-95.

Zaslayskaia, L. A., J. C. Lippmeier, et al. (2001). “Trophic conversion of an obligate photoautotrophic organism through metabolic engineering.” Science 292(5524): 2073-5.

1. A method of modifying a algal cell comprising:
(a) Selecting a selectable marker gene within the genome of a cell which encodes a protein rendering a cell sensitive to a toxic substrate;
(b) Providing a nuclease which specifically recognizes and cleaves a target sequence within said selectable marker gene;
(c) Introducing said nuclease into a cell such that said nuclease cleavage inactivates said selectable marker gene;
(d) Culturing said cell with said toxic substrate and;
(e) Selecting cells which are resistant to the toxic substrate.

2. The method of claim 1 comprising in step c) transforming said cell with a polynucleotide encoding said nuclease and expressing said nuclease into the cell.

3. The method of claim 1 or 2 wherein said algal cell is a diatom.

4. The method of claim 3 wherein said diatom is selected from the group consisting of: Thalassiosira pseudonana or Phaeodactylum tricornutum

5. The method according to any one of claims 1 to 4 wherein said selectable marker gene is the uridine-5′-monophosphate synthase (UMPS) gene and said toxic substrate is the 5-Fluoroorotic acid (5-FOA).

6. The method according to any one of claims 1 to 4 wherein said selectable marker gene is the nitrate reductase gene and said toxic substrate is chlorate.

7. The method according to any one of claims 1 to 4 wherein said selectable marker gene is the tryptophane synthase gene and said toxic substract is 5-fluoroindole.

8. The method according to any one of claims 1 to 7 wherein said nuclease is selected from the group consisting of: TALE-nuclease, MBBBD-nuclease, homing endonuclease, Cas9 nuclease.

9. The method according to any one of claims 1 to 8 further comprising: introducing into a cell a donor matrix comprising at least one homologous region to a part of said selectable marker gene such that said donor matrix recombine with said selectable marker gene.

10. The method according to any one of claims 1 to 9 further comprising introducing at least another protein of interest into said cell.

11. The method of claim 10 wherein said another protein of interest is a nuclease capable of recognizing and cleaving a target sequence of interest.

12. A nuclease which recognizes a target sequence within a gene selected from the group consisting of: the UPMS, nitrate reductase gene and tryptophane synthase.

13. A nuclease which recognizes the target sequence comprised in a nucleic acid sequence selected from the group of: SEQ ID NO: 1 to SEQ ID NO: 4.

14. The nuclease of claim 12 or 13 which is a TALE-nuclease.

15. The TALE-nuclease of claim 14 with an amino acid sequence having at least 70%, 80%, 90%, 95% identity with the amino acid sequence SEQ ID NO: 5 to SEQ ID NO: 8.

16. A polynucleotide encoding the nuclease according to any one of claims 12 to 15.

17. A vector comprising the polynucleotide of claim 16.

18. A kit which comprises a polynucleotide encoding a nuclease capable of recognizing and cleaving a sequence within the UMPS gene and a substrate comprising 5-Fluoroorotic acid (5-FOA).

19. A kit which comprises a polynucleotide encoding a nuclease capable of recognizing and cleaving a sequence within the nitrate reductase gene and a substrate comprising chlorate.

20. A kit which comprises a polynucleotide encoding a nuclease capable of recognizing and cleaving a sequence within the tryptophane synthase gene and a substrate comprising 5-fluoroindole.

21. The kit according to any one of claims 18 to 20 comprising the polynucleotide of claim 16.

22. A diatom which comprises a nuclease according to any one of claims 12 to 15.


SEQUENCE LISTING (17 sequences)

SEQ ID NO: 1 - DNA, 1557 bp, Phaeodactylum tricornutum
atggccaccccctcttttcgatcaaagcttgaagctcgagtcgccgcagtcaactctctcttgtgcgttggtctagacccgcacgagaaagagctgtttgcagacggatgggaaggcgtgccggaagaaaatcgctgtgacgcggcctttaccttttgcaaaacgttggtcgacgcaacattgccttacacggcctgctacaaacccaatgctgcctttttcgaggcgttaggcgatggagggatggcggttctgcgacgagtttgtcaaaacataataccggatgatgtgccgattttgttggatgtcaagcgcggcgacattggctcgaccgctgcggcctacgccgaagcgtgctatggtttgggtgcagactgtgtcacgctttcaccactgatgggatgggactcagtcagtccctttgttacagaaaagtacgttcacaaaggagcatttttgctgtgcaaaacgtcaaatcctggatccaacgattttttagctctgggattacgttcaaatgaatgtttatacgaaagaattgccaagcttgttggctcggaatgggctcagcagaccgagagttcattgggactcgttgtcggggccacagatccagtggccttgtccaaagcgagaaaggctgcaggcgacgacacctggattctagcacccggcgttggtgctcaaggtggagatcttctagaagcagcgcaggctggattgaatacaaaggggacttgcatgctaattcccgtgtctaggggtatcagcaaagctacggacccagcgcaggctgcaaaagaattgcaggagaggattcagaaagctcgggaccaagtcgtggccgcacacatgataaaaaagagttcagacgaagatattaaactctatcaacgcgagtttcttgaatttagtctgtctcaaggtgttctcaaattcggctcttttgtgctgaaaagcggccgcacctctccatattttttcaacgccggtctttttgcttctggcgctgcgttaagcaagcttgggaaagcctatgcttcgactatcatgtcctcggaattattagctgctgggcccaaccaagtcaattttgatgtgatttttggtcctgcatacaagggtatttctctaggtgctgtcgttggaagcgctctgtataacgattttgaagtagatgtcggttttgcgtatgaccgaaaagaggcaaaggatcatggggaaggtggtaaattggtcgggacttcgttggaaggaaaacgagttctgattgtagatgacgtaatcacagcgggaaccgccattcgtgagtcgcacactttgctcaacgatgtgggtgctttgccagttggagtagttattgccctcgatcgagccgaaattcgctctatggaggacaagatttccgctgttcaagcagtcgcacgagatctatctcttctggtcgtgtcaattgtcagtcttcctcaactacagacgtttctcgaacgaagtccggactacggcgatgaaacgctggaaaaagtaactaagtatcgaaacgaatacggagtgtaa

SEQ ID NO: 2 - DNA, 2733 bp, Phaeodactylum tricornutum
  misc_feature (2619)..(2619): n is a, c, g, or t
atggtaccgaaacctgaagatcccacagtcaaggcagagaacaatgcggcgatggatcaacttagtctcctcgacaaagaagatatatcgtcggcttctcgctcgtgccgagaactctacggaccttaccccaaagctattcctgtgccgttcttgaattctcgtaacgaagctcgcgaaggtgacactcccgccgccagcgtcatcgcgcaagccaaaaccatctttgacgtaccggcggactatcgtgacgtgggaacaccggatgaatgggttccccgcgatggacgcctcgtgcgtctgacgggtaagcatcccctcaacgtcgaaccaccgctggcgattctgaagcagcatcgatttattacgccgtcctcgttgcattacgtacgcaaccatggagcgtgcccgaagctgtcttggaaacaacacactgtttgtgtgggaggaaaactggtaccgaatgccttggagctctcgatggacgaaatcgtagcgatggaaccgcgagagctgcccgtcacgttggtctgtgccggaaatcgtcggaaggaacaaaacatgatccgtcaaacaatcggcttcaactggggcccgagcggcgtctcaaccagcgtttggaagggagtgctcctacgcgatttgttgctccgcgcaggggtttcggaaaagaacatggcagggaagcacgtcgaatttattggtgtcgaagacttgccgaacaaggtgggacccgggccgttccaggaggaaccatggggcaaacttgtcaagtacggaaccagtgtcccgctcgctcgggctatgaatccagcgtacgacatcctcattgcctatgagcagaacggcgaagtcttgcagcccgatcacgggtaccccgtccgtctcatcattcctggttatattggaggacggatgattaaatggcttaaatacatcaacgtgattccgcacgaacccaagaatcactatcattaccacgacaatcgcattttacagggaggttggtggtacaaaccggagtacattttcaatgaactcaacatcaattcggccatcgcggctcctgatcacaatgaaacgctttcgatcgccaagaatattgccaagacgtatgacgttacgggttacgcatatactggtggtggtcgtctcatcaccagggtcgaaatttcagttgatggcggtatccattgggaacttgccaaacgtgaacgcaaggagcagccaacggactacggaatgtactggtgctggacttggtggaactacgaagtaaaggtggccgacttggtgggagccaaggaaattatatgccgcgcctgggatgagtccaacaaccctcagccagttgttccaacatggaatctgatgggtatggggaataatcaagcctttcgtgtcaaggtacacatggacaagacagctagcggcgagcatgtgtttcggtttgagcatccaactcagcctggtcaacaaactggtgggtggatgacaaaggtcgccaccaagcctgagtcggccgggttcggacggttgctggaagtgcaggctgagtccaaagaagacgcggccccggctccacctccgaaggaaaataccaaaattttcacgatggaagagattgaaaagcacaacactgaagaagactgttggattgtggtgaaggatcgtgtctacgactgtaccgagtatctagagctgcaccctggcggcattgactcgattgttatcaacggcggcgcagattccacggaagactttgtggcaatccactctaccaaggctacaaagatgctcgagaagtactacattggccagctcgacaaaagtagtgtggccgaggagaaaaaacaagaagacgaacctctcgtcgatgccgatggcaatgctcttgccttgaacccaaagaagaagacgccatttcgtctccaaaacaaaatcacacttagtcgagacagctacctattggattttgctttgccaagcccaaagcatgttttggggctacccacgggaaagcacatgtttatttcggccctcattaatggagagatggtactccggcgctacactcctatctcatccaattacgacattggatgtgtaaagtttgttgtcaaggcataccgtccgtgtgaacgctttccagacggtggcaagatgagccaatacctagaccagatcaatgttggcgactatgttgatatgcgcggaccagttggggaatttgagtactcggccaacggcagttttacaatcgacgccgaaccttgttttgccaccaggttcaacatgcttgctggggggaccggcataacgcccgtaatgcagattgctgcggaaattttgcgaaacccacaagaccctacacaaatgtcccttatttttgcatgccgcgaggaaggcgatctcttgatgcgaagcactttggacgaatgggctgctaactttcctgacaagttcaagattcactacatcctatctgacagctggtcttccgactggaagtattccacaggattcgtagacaaagcgctattttccgagtacttgtacgaagcaggcgataatgtttacagcctcatgtgcggcccaccaattatgttagagaaaggntgccgtccaaacttgggagagccttggtcacaaaaaggacaaaattttttccttttaaaagttcttggactgattgtcatatcaattttgcactttacaatacattttcaatag

SEQ ID NO: 3 - DNA, 49 bp, Phaeodactylum tricornutum
tttagtctgtctctaggtgttctcaaattcggctcttttgtgctgaaaa

SEQ ID NO: 4 - DNA, 49 bp, Phaeodactylum tricornutum
tgaagcagcatcgatttattacgccgtcctcgttgcattacgtacgcaa

SEQ ID NO: 5 - protein, 1088 aa, artificial sequence
  UMPS-TALEN-T01-L1
MGDPKKKRKVIDYPYDVPDYAIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAA

SEQ ID NO: 6 - protein, 1094 aa, artificial sequence
  UMPS-TALEN-T01-R1
MGDPKKKRKVIDKETAAAKFERQHMDSIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAA

SEQ ID NO: 7 - protein, 1088 aa, artificial sequence
  NR-TALEN-T02-L2
MGDPKKKRKVIDYPYDVPDYAIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 8 - protein, 1094 aa, artificial sequence
  NR-TALEN-T02-R2
MGDPKKKRKVIDKETAAAKFERQHMDSIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 9 - DNA, 3267 bp, artificial sequence
  UMPS-TALEN-T01-L1
atgggcgatcctaaaaagaaacgtaaggtcatcgattacccatacgatgttccagattacgctatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgcttga

SEQ ID NO: 10 - DNA, 3285 bp, artificial sequence
  UMPS-TALEN-T01-R1
atgggcgatcctaaaaagaaacgtaaggtcatcgataaggagaccgccgctgccaagttcgagagacagcacatggacagcatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgcttga

SEQ ID NO: 11 - DNA, 3270 bp, artificial sequence
  NR-TALEN-T02-L2
atgggcgatcctaaaaagaaacgtaaggtcatcgattacccatacgatgttccagattacgctatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 12 - DNA, 3288 bp, artificial sequence
  NR-TALEN-T02-R2
atgggcgatcctaaaaagaaacgtaaggtcatcgataaggagaccgccgctgccaagttcgagagacagcacatggacagcatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 13 - DNA, 5922 bp, artificial sequence
  plasmid vector pCLS20603
gggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtaccataactagttcggcgcgccaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaaccatgggcgatcctaaaaagaaacgtaaggtcatcgattacccatacgatgttccagattacgctatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgcttgataactcgagcgatcctctagacgagctcctcgagcctgcagcagctgaagctttaagatccaatggcaaggaccaagtgctggaacttgttttgctttagcagatctagatcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcatatcctgca

SEQ ID NO: 14 - DNA, 5940 bp, artificial sequence
  plasmid vector pCLS20604
gggtacgtttaaacgtattaattaagacctagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttggtaccataactagttcggcgcgccaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaaccatgggcgatcctaaaaagaaacgtaaggtcatcgataaggagaccgccgctgccaagttcgagagacagcacatggacagcatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgcttgataactcgagcgatcctctagacgagctcctcgagcctgcagcagctgaagctttaagatccaatggcaaggaccaagtgctggaacttgttttgctttagcagatctagatcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcatatcctgca

SEQ ID NO: 15 - DNA, 6456 bp, artificial sequence
  plasmid vector pCLS16353
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgatgcatccgttaacaccggtaagcggccgcgctagggataacagggtaatattcaaaacgtcgtacgacgttttgacctgcaggaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaattaattaagtaggcgcgccactagcgctgtcacgcgccaagccgccaccatgggcgatcctaaaaagaaacgtaaggtcatcgattacccatacgatgttccagattacgctatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctctagcttgagctctcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcagtttaaactcactgactcgctgcgctcggtcgttcggctgcggcgagcggtatcagctcactcaaaggcggtaatacggttatccacagaatcaggggataacgcaggaaagacaattgcttataacacgcgtactagtgctcgcgacgagatcttacttaagcagtcgacaacctaggattagcgctccggtacctcaaaacgtcgtacgacgttttgagctagggataacagggtaatatggatccaagatatcaagaattcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 6474 bp, artificial sequence
  plasmid vector pCLS16354
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgatgcatccgttaacaccggtaagcggccgcgctagggataacagggtaatattcaaaacgtcgtacgacgttttgacctgcaggaatctcgcctattcatggtgtataaaagttcaacatccaaagctagaacttttggaaagagaaagaatgtccgaatagggcacggcgtgccgtattgttggagtggactagcagaaagtgaggaaggcacaggatgagtttcctcgagacacatagcttcagcgtcgtgtaggctaggcagaggtgagttttctcgagacataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccagctacgacgctcgccccagctggggcttccttactatacacagcgcatatttcacggttgccagaattaattaagtaggcgcgccactagcgctgtcacgcgccaagccgccaccatgggcgatcctaaaaagaaacgtaaggtcatcgataaggagaccgccgctgccaagttcgagagacagcacatggacagcatcgatatcgccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctagacgagctcctcgagcctgcagcagctgaagctctagcttgagctctcgagctacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcagtttaaactcactgactcgctgcgctcggtcgttcggctgcggcgagcggtatcagctcactcaaaggcggtaatacggttatccacagaatcaggggataacgcaggaaagacaattgcttataacacgcgtactagtgctcgcgacgagatcttacttaagcagtcgacaacctaggattagcgctccggtacctcaaaacgtcgtacgacgttttgagctagggataacagggtaatatggatccaagatatcaagaattcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 5428 bp, artificial sequence
  plasmid vector pCLS0003
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc